(19) United States
(12) Patent Application Publication
(10) Pub. No.: US 2006/0069358 A1
(43) Pub. Date: Mar. 30, 2006

(54) Controlled drop dispensing units

(76) Inventor: Scott J. Gerondale, Mission Viejo, CA (US)

Correspondence Address: WALTER A. HACKLER, Ph.D.;PATENT LAW OFFICE, SUITE B, 2372 S.E. BRISTOL STREET, NEWPORT BEACH, CA, 92660-0755, US

(21) Appl. No.: 11/225,486

(22) Filed: Sep. 13, 2005

Related U.S. Application Data
(60) Provisional application No. 60/614,125, filed on Sep. 29, 2004.
(60) Provisional application No. 60/614,197, filed on Sep. 29, 2004.

(51) Int. Cl. A61M 35/00

(52) U.S. Cl. 604/298; 604/300

(57) ABSTRACT

Controlled drop dispensing tube apparatus includes a tube for containing a medicament, a dispensing tip having an aperture in fluid communication with the tube, a collapsible shell for containing the tube, and a mechanism disposed in the shell for comprising the tube in order to force medicament from the tube and through the dispensing tip upon collapsing of the shell.

Ophthalmic treatment fluids are commonly administered to the eye through the use of squeezable eyedropper bottles.

This type of application is accompanied with a number of disadvantages such as discomfort from the application of the drop and involuntary blinking. In addition, an excessive fluid results in a portion of the drop running onto the skin and about the eye. In fact, only a small amount of a 50 microliter drop is effective with the remainder lost by drainage either externally, as hereinabove noted, or through nasolacrimal drainage.

This, of course, is wasteful and impedes the effectiveness of treatment.

In addition to the occasional administration of eye drops, many patients such as those with glaucoma require continued use of eye drops. Accordingly, the necessity of continued eye drop administration usually requires the drops to be self-administered and the present invention is particularly useful for this purpose.

### SUMMARY OF THE INVENTION

A controlled drop dispensing unit in accordance with the present invention generally includes a cavity for containing a medicament along with an aperture in fluid communication with the cavity and a manual operated trigger. A mechanism is provided for forcing a metered drop of medicament from the cavity and through the aperture upon each operation and trigger.

More specifically, the cavity may comprise a syringe and a needle may be provided having a lumen in fluid communication with the syringe with the aperture disposed at a needle end.

The mechanism further includes a plunger disposed in the syringe with the plunger including a rack shaft and a gpawl for intermittently engaging the rack shaft.

A spring is provided for driving the plunger and the manually operated trigger includes a button interconnecting with the pawl for intermittently engaging the pawl with the rack shaft.

More particularly, a unit may include a nasal bridge positioning guide and a port surrounding the needle end. In addition, the mechanism may include a plunger disposed in the syringe with the plunger including a rack and the mechanism further including a pinion gear for engaging the rack and the pawl for intermittently engaging the pinion gear.

In one embodiment, a manually operated trigger includes a rotary knob for intermittently disengaging the pawl from the pinion gear.

An eyeglass shaped frame may be provided for supporting the needle.

In another embodiment, the cavity includes an elastomeric bladder with a mechanism comprising a valve body with a valve tappet and valve syringe and the trigger includes a valve interconnected with the valve tappet by a sear for intermediately engaging a valve skirt for releasing a spring in order to force the drop through a nozzle in fluid communication with the aperture.

In yet another embodiment of the present invention, the cavity may include a vial with a floating piston disposed therein in a syringe in fluid communication with the vial via a manifold and a duckbill check valve. The mechanism includes a plunger disposed in the syringe and a spring for moving the plunger and the trigger comprises a lever for intermittently engaging the spring for intermittently forcing the metered drops of medicament from the syringe and vial through the aperture.

In still another embodiment of the present invention, the cavity may include a rotary chamber disposed in a housing and a mechanism includes a main spring for rotating the chamber to expel medicament through the aperture and the trigger disposed for rotational cocking of the main spring and releasing the main spring by way of a catch.

In further embodiment of the present invention, the cavity may include a plurality of blisters and the mechanism comprises a hammer for sequentially rupturing each blister and forcing the metered drops of medicament from the blister through the aperture. In this embodiment, the blisters are preferably arranged on a tape and the mechanism further includes a sprocket for advancing the tape in order that the blisters are sequentially aligned with the hammer.

In still another embodiment of the present invention, the cavity is disposed in a tube, the aperture is disposed in a dispensing tip, and the unit further comprises a collapsible shell for containing the tube. The mechanism is disposed in the shell for compressing the tube in order to force medicament from the tube and through the dispensing tip aperture upon collapse of the shell.

More particularly, the shell may include an upper portion and a lower portion with the upper portion being slidably received by the lower portion. The mechanism may include a roller for flattening the tube as the upper shell portions are forced into the lower shell portion.

Preferably, the mechanism includes gears attached between the ends of the roller and the interior surfaces of the lower portion for rotating the roller as the upper shell portions are forced into the lower shell portion. In addition, the mechanism may include racks disposed on an exterior surface of the upper portion and engaging the gears for rotating the gears as the upper shell portions is forced into the lower shell portion.

More particularly, the upper shell portion may include finger grooves and the lower shell portion may include a thumb groove for enabling the upper shell portion to be forced into the lower shell by one hand of the user. Preferably, the upper shell portion includes observable scale indicia for exhibiting dosage dispensed as a function of the upper shell portion position within the lower shell portion.

In that regard, the lower shell portion may include a window for observing the scale indicia. Preferably, the dispensing tip aperture is disposed at an angle with the longitudinal axis of the tube and the tube includes an aperture position guide including a support member and circling the aperture at a spaced apart distance therefrom.

### BRIEF DESCRIPTION OF THE DRAWINGS

The advantages and features of the present invention will be better understood by the following description when considered in conjunction with the accompanying drawings, in which:

FIG. 1 is a perspective view of one embodiment of the present invention as it may be utilized for dispensing drops into a users eyes including a nasal bridge positioning guide along with a button for activating a dispensing of drops into a users eye;

FIG. 2 is a cross sectional view of the embodiment shown in FIG. 1;

FIG. 3 is an alternative of the present invention utilizing an eyeglass shaped frame along with a rotary dosing knob;

FIG. 4 is an enlarged perspective view of the mechanism operated by the rotary dosing knob;

FIG. 5 is a cross sectional view of another embodiment of the present invention utilizing an elastomeric bladd4er;

FIG. 6 is a cross sectional view of yet another embodiment of the present invention generally including a vial, floating piston, duckbill check valve, plunger, and trigger along with a soft eye cup;

FIG. 7 is an elevation view of further embodiment in accordance with the present invention utilizing a rotary chamber cocking trigger and nozzle;

FIG. 8 is a perspective view of still another embodiment of the present invention;

FIG. 9 is a perspective view of a mechanism disposed in the embodiment shown in FIG. 8;

FIG. 10 is another perspective view of the mechanism shown in FIG. 9;

FIG. 11 is a perspective view of further embodiment of the present invention generally illustrating a collapsible shell;

FIG. 12 is a broken view of the embodiment shown in FIG. 11 illustrating a mechanism for collapsing of a shell along with a tube disposed therein for containing a medicament;

FIG. 13 is a perspective view of an alternative embodiment of a tube for use in the embodiment shown in FIGS. 11-12; and

FIG. 14 is yet another embodiment of the present invention illustrating a tip and aperture disposed within an aperture positioning guide and a one piece blow molded tube.

### DETAILED DESCRIPTION

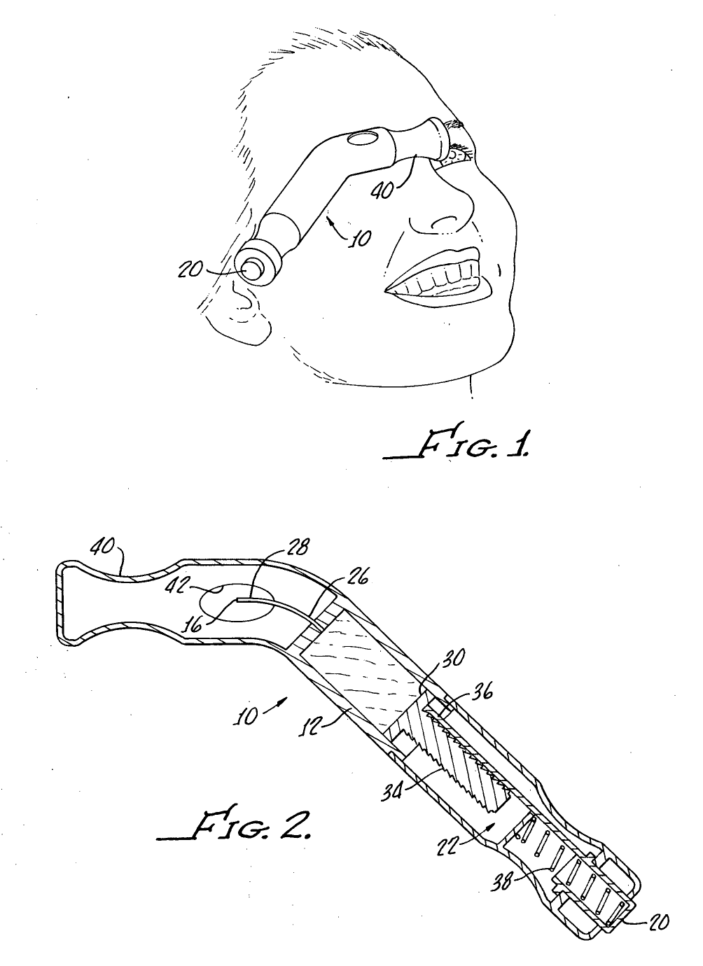

With reference to FIGS. 1 and 2, there is shown a controlled drop dispensing unit 10 in accordance with the present invention, generally including a cavity/syringe 12 for containing a medicament (not shown), a needle aperture 16 in fluid communication with the cavity 12, and a trigger/button 20 for manually operating a mechanism 22 for forcing metered drops (not shown) of medicament from the cavity 12 through the aperture 16 upon each operation of the trigger 20.

More particularly, a needle 26 may be provided having a lumen (not shown) in fluid communication with the syringe 12 and the aperture 16 is disposed in a needle end 28. In addition, the mechanism 22 may include a plunger, or piston, 30 disposed in the syringe 12 with the plunger 30 including a rack shaft 34 and the mechanism 22 further includes a pawl 36 for intermittently engaging the rack shaft 34 with a spring 38 for driving the plunger 30. The manually operated trigger/button 20 is interconnected with the pawl 36 for intermittently disengaging the pawl 36 from the rack shaft 34.

Further, a nasal bridge positioning guide 4d may be provided along with a port 42 (see FIG. 2) surrounding the needle end 28 for convenience of the user.

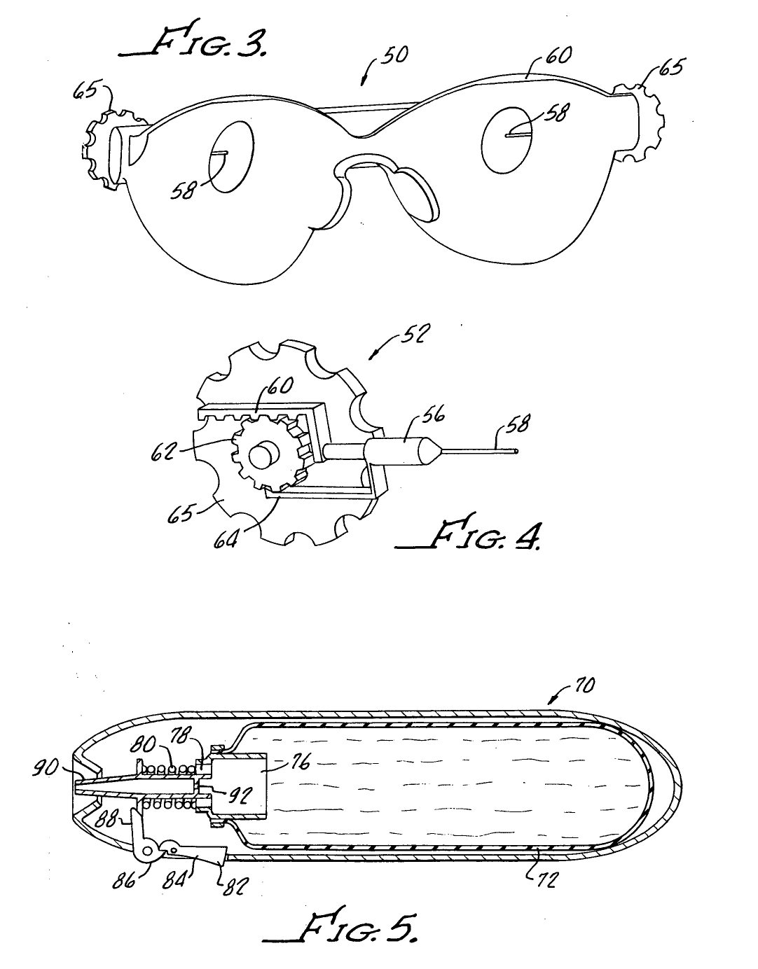

In another embodiment 50 of the present invention, shown in FIGS. 3 and 4, the mechanism 52 may include a plunger 54 disposed in a syringe 56 having a needle 58. The plunger 54 includes a rack 60 and the mechanism 52 further includes a pinion gear 62 for engaging the rack 60 and a pawl 64 for intermittently engaging the pinion gear 62. In this embodiment 50, a manually operated trigger rotary knob 65 for intermittently disengaging the pawl 64 from the pinion gear 62. For convenience of use, this embodiment may further include eyeglass shaped frame 68 for supporting the needle 58.

In yet another embodiment 70 of the present invention, shown in FIG. 5, an elastomeric bladder 72 provides a cavity with a mechanism 74 comprising a valve body 76 with a valve tappet 78 and a valve spring 80 and a trigger 82 includes a lever 84 interconnecting the valve tappet 78 by a sear 86 for intermittently engaging a valve skirt 88 for releasing the spring 80 for forcing a medicament through a nozzle 90 which includes an aperture 92 in fluid communication with the bladder 72.

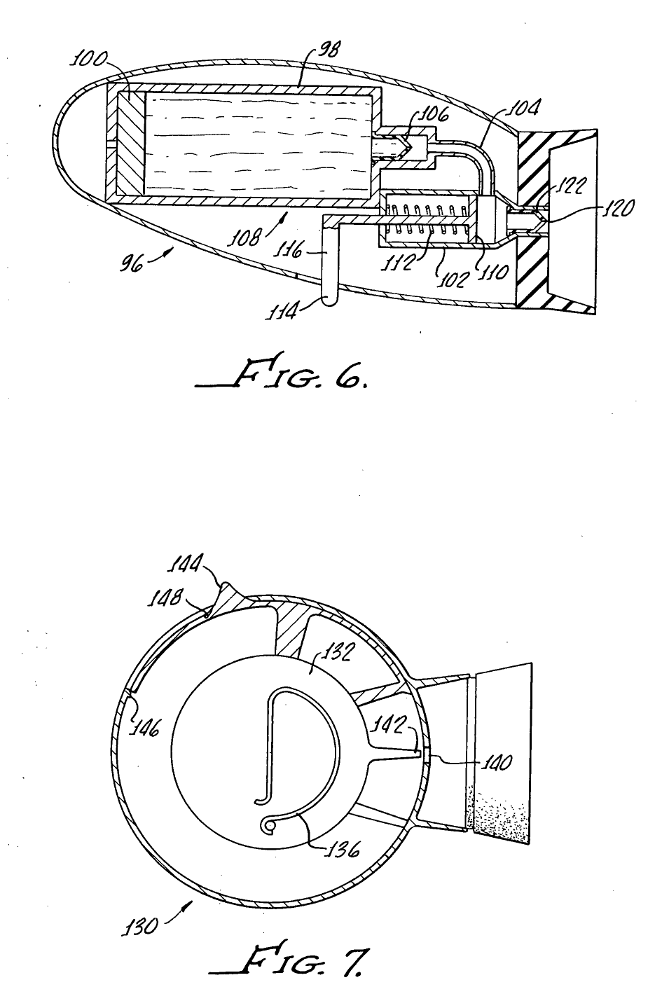

In still another embodiment 96 of the present invention, shown in FIG. 6, a vial 98 provides a cavity with a floating piston 100 disposed therein and a syringe 102 in fluid communication with the vial 98 via a manifold 104 and a duckbill check valve 106.

A mechanism 108 further comprises a plunger 110 disposed in the syringe 102 and a spring 112 is provided for moving the plunger 110 with a trigger 114 comprising a lever 116 for intermittently engaging the spring 112 for forcing metered drops of medicament from the syringe 102 and the vial 98 through an aperture 120 and check valve 122.

In a further embodiment 130 of the present invention, shown in FIG. 5, a rotary chamber 132 provides a cavity which is disposed in a housing 134 and a main spring 136 provides a mechanism for rotating the chamber 132 to expel medicament (not shown) through an aperture 140 in a nozzle 142. A trigger 144 is disposed for rotatably cocking the main spring 136 and releasing the main spring 136 by way of a catch 146 and notch 148.

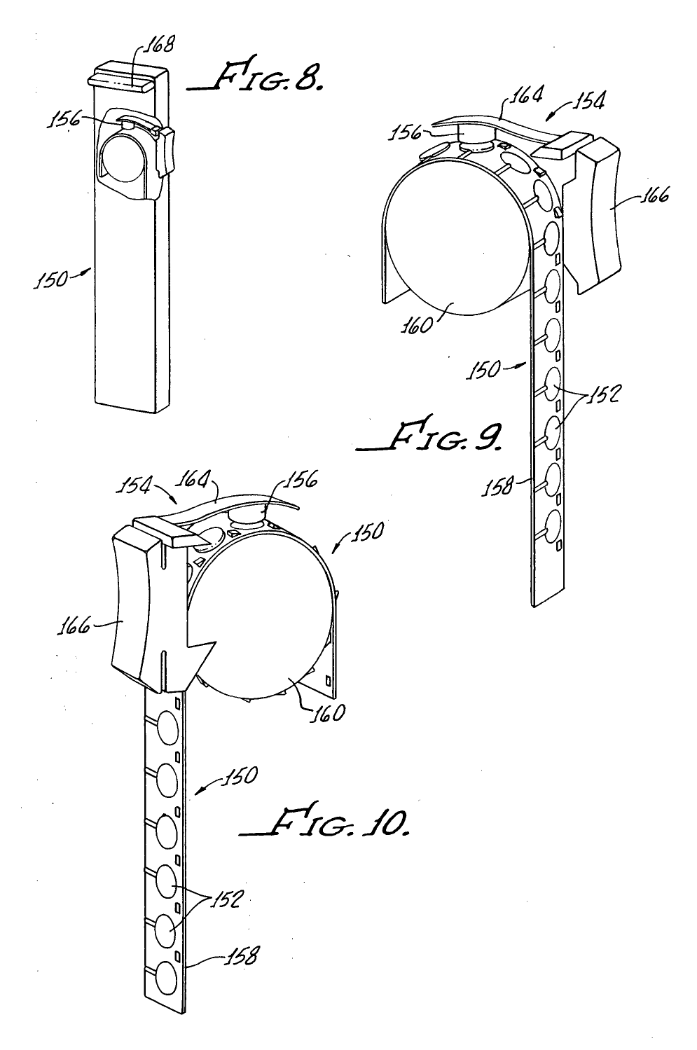

And still yet another embodiment 150 in accordance with the present invention, shown in FIGS. 8-9, a plurality of blisters 152 provide cavities and a mechanism 154 comprises a hammer 156 for sequentially rupturing each blister 152 and forcing the metered drops of medicament from the blister 152 through an aperture 156 by way of nozzle 157 formed in a tape 158.

In this embodiment 150, the blisters 152 may be arranged on the tape 158 and the mechanism 154 further comprises a sprocket 160 for advancing the tape 158 in order that the blisters 152 are sequentially aligned with the hammer 156 for rupture thereof. A hammer spring 164 links the hammer 156 with a trigger 166. A brow guide 168 may be provided to provide spaces between the aperture 156 and an eye (not shown).

An alternative controlled drop dispensing unit 200 in accordance with the present invention is shown in FIGS. 11-13 which generally includes a tube 202 for containing a medicament (not shown) and a dispensing tip 204 having an aperture 206 in fluid communication with the tube.

A collapsible shell 210 is provided for containing the tube 202 and a mechanism 212 disposed in the shell 210 is provided for compressing the tube 202 in order to force a medicament from the tube 202 and through the dispensing tip 204 upon collapse of the shell 210.

More particularly, the shell includes an upper portion 216 and a lower portion 218 with the upper portion 216 being slidably received by the lower portion 218. In addition, the mechanism 212 includes a roller 220 for flattening the tube 202 as the upper shell portion 216 is forced into the lower shell portion 218.

More particularly, the mechanism 212 for compressing the tube 202 includes gears 220, 222 attached between ends of the roller 220 and interior surfaces 230, 232 of the lower portion 218 for rotating the roller 220 as the upper shell portion 216 is forced into the lower shell portion 218.

In addition, racks 240 are provided and disposed on an interior surface of the upper portion 218 for rotatably engaging the gears 220, 222 as the upper shell portion 216 is forced into the lower shell portion 218.

Compression of the shell 210, that is, forcing of the upper portion 216 into the lower portion 218, is enabled through the use of finger grooves 250 on the upper shell portion 216 and a thumb groove 252 on the lower shell portion 218. Placement of these grooves 250, 252 enables the forcing of the upper portion 216 into the lower portion 218 with one hand.

In addition, the upper shell portion 218 may include scale indicia 256 for exhibiting dosage dispensed as a function of the upper shell portion 216 position within the lower shell portion 218. In that regard, a window 258 may be provided in the lower shell portion 218 for observing the scale indicia 256.

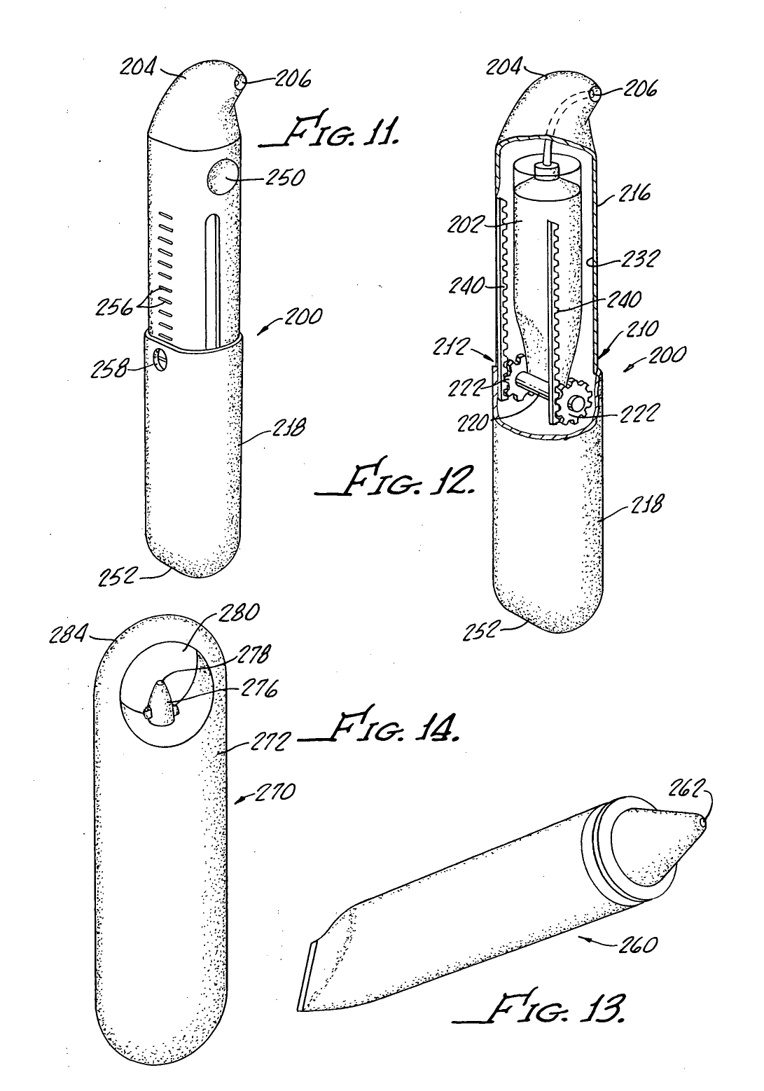

In the embodiment 200 shown in FIGS. 11-12, the aperture 206 is disposed at an angle with a longitudinal axis of the tube.

An alternative tube embodiment 260 of the present invention is shown in FIG. 13 in which the aperture tip 262 and tube are aligned along a single longitudinal axis.

With reference to FIG. 14, there is shown an alternative embodiment 270 including a tube 272 in accordance with the present invention for use with the upper and lower portions 216, 218 hereinabove described in which a tip 276 includes an aperture 278 positioning guide 280 includes a support member 284 which encircles the aperture 278 at a spaced apart distance therefrom and enables portion of the aperture 278 over an eye (not shown).

Although there has been hereinabove described a specific controlled drop dispensing units in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A controlled drop dispensing unit comprising:
a cavity for containing a medicament;
an aperture in fluid communication with said cavity;
a manually operated trigger; and
a mechanism for forcing a metered drop of medicament from said cavity and through said aperture upon each operation of the trigger.

2. The unit according to claim 1 wherein said cavity comprises a syringe.

3. The unit according to claim 2 further comprises a needle having a lumen in fluid communication with said syringe and said aperture is disposed at a needle end.

4. The unit according to claim 3 wherein said mechanism includes a plunger disposed in said syringe, said plunger including a rack shaft and said mechanism further includes a pawl for intermittently engaging said rack shaft, a spring for driving said plunger and said manually operated trigger includes a button interconnected with said pawl for intermittently disengaging said pawl with said rack shaft.

5. The unit according to claim 4 further comprises a nasal bridge positioning guide and a port surrounding the needle end.

6. The unit according to claim 3 wherein said mechanism includes a plunger disposed in said syringe, said plunger includes a rack and said mechanism further includes a pinion gear for engaging said rack and a pawl for intermittently engaging said pinion gear.

7. The unit according to claim 6 wherein said manually operated trigger includes a rotary knob for intermittently disengaging said pawl from said pinion gear.

8. The unit according to claim 7 further comprising an eyeglass shaped frame for supporting said needle.

9. The unit according to claim 1 wherein said cavity comprises an elastomeric bladder, said mechanism comprises a valve body with a valve tappet and valve syringe, and the trigger includes a valve interconnected to said valve tappet by a sensor for intermittently engaging a valve skirt for releasing a spring for forcing the drop through a nozzle in fluid communication with said aperture.

10. The unit according to claim 1 wherein said cavity comprises a vial with a floating piston disposed therein and a syringe in fluid communication with said vial via a manifold and a duckbill check valve, said mechanism comprises a plunger disposed in said syringe and a spring for moving said plunger and the trigger comprises a lever for intermittently engaging said spring for intermittently forcing the metered drops of medicament from said syringe and vial through the aperture.

11. The unit according to claim 1 wherein said cavity comprises a rotary chamber disposed in a housing and said mechanism comprises a mainspring for rotating the chamber to expel medicament through the aperture and the trigger is disposed for rotational cocking of said mainspring and releasing the mainspring by way of a catch.

12. The unit according to claim 1 wherein said cavity comprises a plurality of blisters and said mechanism comprises a hammer for sequentially rupturing each blister and forcing the metered drops of medicament from the blister through said aperture.

13. The unit according to claim 12 wherein said blisters are arranged on a tape and the mechanism further comprises a sprocket for advancing the tape in order that the blisters are sequentially aligned with the hammer.

14. The unit according to claim 1 wherein said cavity is disposed in a tube, the aperture is disposed in a dispensing tip and further comprising a collapsible shell for containing said tube, said mechanism is disposed in said shell for compressing said tube in order to force medicament from said tube and through the dispensing tip aperture upon collapse of said shell.

15. The unit according to claim 14 wherein the shell includes an upper portion and a lower portion, said upper portion being slidably received by said lower portion.

16. The unit according to claim 14 wherein said mechanism comprises a roller for flattening said tube as the upper shell portion is forced into the lower shell portion.

17. The unit according to claim 16 wherein said mechanism further comprises gears attached between ends of said roller and interior surfaces of the lower portion for rotating said roller as the upper shell portion is forced into the lower shell portion.

18. The unit according to claim 17 wherein said mechanism further comprises racks disposed on an interior surface of the upper portion end and engaging said gears for rotating said gears as the upper shell portion is forced into the lower shell portion.

19. The unit according to claim 18 wherein the upper shell portion includes finger grooves and the lower shell portion includes a thumb groove for enabling the upper shell portion to be forced into the lower shell by one hand of a user.

20. The unit according to claim 19 wherein the upper shell portion includes observable scale indicia for exhibiting dosage dispensed as a function of the upper shell portion position within the lower shell portion.

21. The unit according to claim 20 wherein the lower shell potion includes a window for observing the scale indicia.

22. The unit according to claim 14 wherein the dispensing tip aperture is disposed at an angle with a longitudinal axis of said tube.

23. The unit according to claim 14 wherein said tube includes an aperture position guide including a support member encircling said aperture at a spaced apart distance therefrom.
